(19) United States
(12) Patent Application Publication
(10) Pub. No.: US 2013/0045865 A1
(43) Pub. Date: Feb. 21, 2013

(54) HIGH ACTIVITY EARLY TRANSITION METAL CARBIDE AND NITRIDE BASED CATALYSTS

(71) Applicants: Levi T. Thompson, Superior Township, MI (US); Neil Schweitzer, Plymouth, MI (US); Joshua Schaidle, Ann Arbor, MI (US)

(72) Inventors: Levi T. Thompson, Superior Township, MI (US); Neil Schweitzer, Plymouth, MI (US); Joshua Schaidle, Ann Arbor, MI (US)

(73) Assignee: THE REGENTS OF THE UNIVERSITY OF MICHIGAN, Ann Arbor, MI (US)

(21) Appl. No.: 13/362,937

(22) Filed: Jan. 31, 2012

Related U.S. Application Data
(60) Provisional application No. 61/437,874, filed on Jan. 31, 2011.

(51) Int. Cl. B01J 27/22; B01J 27/24; C07C 1/04; C01B 3/06

(52) U.S. Cl. 502/177; 423/656; 502/185; 502/184; 423/655; 518/713; 518/715; 518/716; 518/714

(57) ABSTRACT

A catalyst composition contains an active metal on a support including a high surface area substrate and an interstitial compound, for example molybdenum carbide. Pt—Mo₂C/Al₂O₃ catalysts are described. The catalyst systems and compositions are useful for carrying out reactions generally related to the water gas shift reaction (WGS) and to the Fischer-Tropsch Synthesis (FTS) process.

### CROSS-REFERENCE TO RELATED APPLICATIONS

This application claims the benefit of U.S. Provisional Application No. 61/437,874, filed on Jan. 31, 2011. The entire disclosure of the above application is incorporated herein by reference.

### GOVERNMENT RIGHTS

This invention was made with U.S. Government support under grant CBET 0933239 awarded by the National Science Foundation. The U.S. Government has certain rights in the invention.

### FIELD

The present technology relates to supported metal heterogeneous catalysts for the water gas shift reaction built on early transition interstitial metal compounds such as carbides and nitrides.

### INTRODUCTION

Hydrogen gas has a number of important industrial applications including, for example, petroleum refining, powering fuel cells, production and processing of chemicals, and manufacturing semi-conductor materials. Hydrogen does not naturally exist as large deposits of hydrogen gas, but is found as part of molecules such as water or hydrocarbons, such as petroleum or coal. Accordingly, hydrogen gas for use in industrial applications is usually produced from water and hydrocarbon starting materials using a series of catalytic steps that generally provide hydrogen gas along with various byproducts, such as oxygen, carbon monoxide, and carbon dioxide.

Various methods for the production of hydrogen convert hydrocarbons, such as alcohol, natural gas, gasoline, or diesel fuel, into a hydrogen rich gas in a series of steps. These steps can include steam reforming or partial oxidation where the hydrocarbon is reacted with water or oxygen to form hydrogen gas along with other by-products, such as carbon monoxide and carbon dioxide. Carbon monoxide may be further reacted with water to yield additional amounts of hydrogen; this is known as the Water-Gas Shift (WGS) reaction. The WGS reaction can be depicted as follows, where carbon monoxide reacts with water vapor to form carbon dioxide and hydrogen: CO_{(g)}+H₂O₍ᵥ₎→CO_{2(g)}+H_{2(g)}. The WGS reaction is generally carried out by passing a reactant gas stream over a solid catalyst in a heterogeneous reaction. For example, the WGS reaction has been used as a method to remove carbon monoxide from reformate in fuel cell applications. The WGS reaction can use two temperature domains or stages. The high temperature shift (HTS) at about 350° C., and the low temperature shift (LTS) at about 190-210° C. Typical catalysts used industrially for these processes include iron oxide (commonly for the HTS process) and copper/zinc oxide (for the LTS process), where both can be used with appropriate promoters and additives.

Various methods employing hydrogen include those using the Fischer-Tropsch Synthesis (FTS) process, which is a set of chemical reactions that convert a mixture of carbon monoxide and hydrogen into liquid hydrocarbons. The FTS process is useful in various gas-to-liquid technologies and can be used to produce petroleum substitutes, such as synthetic lubricants and synthetic fuels, typically using hydrogen generated from coal, natural gas, or biomass. The FTS process involves a series of chemical reactions that can lead to a variety of hydrocarbons. For example, alkanes can be produced according to the equation: (2n+1) H₂+nCO→CₙH₍₂ₙ₊₂₎+nH₂O; where n is a positive integer. The WGS reaction can be used in conjunction with the FT reaction to vary the H2/CO ratio of the reactant gas stream.

Conversion rates of reactants and overall yields of products in such methods are dependent on the function and the nature of the catalyst(s) employed. Likewise, the size, weight, and cost of systems used to generate hydrogen depend on the efficiency of the catalysts used for the WGS reaction, FTS reaction, and/or other reactions employed in the overall process.

Heterogeneous catalysts and related materials generally include catalytically active materials added to supports with high surface areas in order to increase the level of interaction between reactants. In many cases, the support comprises an inorganic oxide selected to have a large surface area per unit weight. One particular example is aluminum oxide or alumina, which provides a relatively large surface area per unit weight. Other particular examples include using naturally existing or synthetic zeolites as catalytic supports. Such materials, though commonly used as catalytic supports for noble metal catalytic systems, present certain limitations. In particular, these support materials are oxide-based materials and oxide surfaces often do not provide an optimum surface for the formation of thin layers of elemental noble metals. In fact, metals in elemental form do not readily wet the surface of most oxides based on surface energy differences. This disadvantageous property commonly leads to the formation of relatively large, symmetric particles sitting on the surface of the support. The particle shapes (e.g., cubo-ocathedral) do not maximize the surface area to volume ratio of metal available for interaction with reactants. This is a particular challenge for expensive noble metals including (in order of increasing atomic number) ruthenium, rhodium, palladium, silver, osmium, iridium, platinum, and gold. Another problem arises due to the fact that noble metals tend to react with contaminants present in feed materials which can lead to poisoning of catalytically active sites and the need to replace the catalytic system, thereby incurring additional expenses.

It is desirable to find new catalytic systems that can bring together a number of different characteristics enveloped within the same family of compounds. Desirable characteristics for a given support include, but are not limited to, high surface area, high temperature stability (refractory) and workability, controllable purity, engineered stoichiometry, and the ability to accept and interact with traditional catalytic metals, such as Ni, Pt, Rh, in a manner advantageous to catalytic processes. One particular system of materials of interest is interstitial transition metal compounds, more specifically hydrides, borides, carbides and nitrides. One problem associated with these particular compounds can be their high affinity to ambient oxygen which can prevent the deposition of catalytic metals directly onto the native interstitial compound.

Interstitial compounds or interstitial alloys are compounds formed when the spaces within atoms in a metallic crystal lattice are sufficiently large as to accommodate the inclusion of atoms fitting within that space to form relatively stable solutions or varied composition entities. Examples of inclusion elements include C giving rise to carbides, N to nitrides, B to borides and H to hydrides respectively. One characteristic of these compounds is that they tend to maintain their metallic nature and bonding characteristics or properties. In addition, by varying the ratio of metal to solute, it is in principle possible to manipulate different engineering properties of the material.

### SUMMARY

In various embodiments the invention provides a concept and synthesis of a type of core-shell catalyst in which a nanosized carbide or nitride particle is the core and a metal acts as the shell. In some embodiments, the catalyst is not strictly speaking a core shell catalyst as that term is more rigorously used to denote a catalyst where a core is completely covered by a metal. Indeed, FIG. 3 shows that a Pt—Mo₂C/Al₂O₃ catalyst has significant catalytic activity even at a 10% surface coverage of Pt.

Although the invention is not to be limited by any theory, a number of observations demonstrate unique features of the system, mentioned here in the context of Pt/Mo₂C:

Using density functional theory (DFT) calculations, it can be shown that the Pt binds stronger to Mo₂C than it does to itself. This leads one to conjecture that in reaction environments which lead to the dissolution/aggregation of platinum (electrochemical systems-fuel cells), the Pt will be less likely to dissolve or aggregate into larger particles because it is bound so strongly to Mo₂C.

Mo₂C and other interstitial compounds are of course much less expensive material than Pt, thus its use as a core material reduces the overall amount of Pt required (and the overall cost).

DFT calculations also support the conclusion that the electronic interactions between Pt and Mo₂C result in the properties of Pt being modified. For example, the adsorption of CO on Pt(111) has a reaction energy of −1.8 eV. However, the adsorption of CO on a Pt monolayer on Mo₂C has a reaction energy of −1.4 eV. These types of electronic effects will modify the reactive of the thin shell layer of Pt, potentially making it more active than bulk Pt.

Ease of synthesis. Typically, the synthesis of core shell catalysts involves complex vacuum coating technologies such as atomic layer deposition and chemical vapor deposition, electrochemical methods in which a voltage is applied to an electrode surrounded by an electrolyte containing the metal precursor in order to drive the formation of a thin layer of metal on the core material, or a borohydride reduction method. It is observed that synthesis of Pt/Mo₂C core shell materials could be carried out via simple wet impregnation due to the ability of the Mo₂C surface to reduce the Pt precursor in an aqueous solution.

In some embodiments, the present technology provides for the preparation of transition metal interstitial compound catalyst supports where the metal interacts directly with the native interstitial component. Another aspect of the present technology is to engineer the ratio of transition metal to the interstitial component thus achieving different physico-chemical-structural properties desirable to optimize the behavior, characteristics, and properties of the supported catalyst. As one particular example, molybdenum carbide is active for the WGS reaction with rates that can be competitive with those for commercial catalysts. In some embodiments, the present technology provides for the preparation, evaluation and characterization of a series of Pt/Mo₂C catalysts with a variety of Pt loadings, where these materials show novel and superior characteristics when compared to other industrial catalysts. This is achieved in part by preparing different loadings of Pt on Mo₂C supports and comparing the WGS rates for these materials to series of analogous oxide supported Pt catalysts.

In some embodiments, the present technology provides novel catalyst compositions comprising a catalyst support material having deposited thereon elemental metals; the catalyst precursor comprising a support selected from a family of metal carbides, nitrides, hydrides, and/or borides. In one particular aspect, molybdenum nitride and molybdenum carbide are used as supports for catalytic compositions. In another aspect, a noble metal precursor is used to form thin layers of noble metal on the surface of the support. Other aspects include using base metals like Fe and Cu as the elemental metal. In one particular case chloroplatinic acid is used as a precursor to elemental platinum thin layers adhered to the support upon treatment. The disclosed catalyst systems and compositions are particularly useful for carrying out reactions generally related to the water gas shift reaction (WGS) and to the Fischer-Tropsch Synthesis (FTS) process.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1a. High resolution HAADF-STEM electron micrographs of a) a Pt particle supported on Mo₂C. FIG. 1b. A Pt particle supported on carbon. FIG. 1c. Intensity line scans for the Pt particle supported on Mo₂C in FIG. 1a and the Pt particle supported on carbon in FIG. 1b.

FIG. 2. HAADF-STEM Electron micrographs of a) 4% Pt/Mo₂C catalyst particle and b) a Pt particle supported on Mo₂C.

FIG. 3a. Arrhenius plots of the WGS reaction. FIG. 3b. WGS rates as a function of Pt loading including predicted rates from the surface site and perimeter site models.

FIG. 4. Particle substrate schematic and perimeter site model.

FIG. 5a. Particle-substrate-adsorbate schematic showing schematic of adsorbate on step site. FIG. 5b. Particle-substrate-adsorbate schematic showing bound CO.

FIG. 6. Particle-substrate schematic with dimensions.

FIG. 7. Core-shell model depicting Mo₂C on high surface area support particle (also referred to herein as a high surface area substrate) and Pt raft-like particles on the surface of the interstitial metal substrate (also referred to herein as the interstitial (metal) compound), (a) transversal cut schematic, (b) spherical particle schematic. FIG. 7 is a schematic drawing, not necessarily to scale, illustrating some of the features and concepts of catalyst compositions wherein a metal like Pt is deposited on a catalyst support made of nanosized “islands” of interstitial compounds attached to a high surface area support particle.

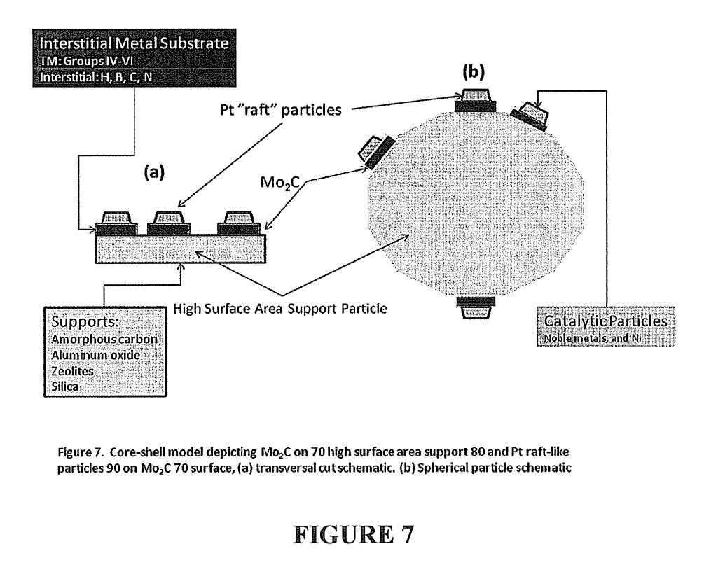

FIG. 8 is a STEM micrograph of molybdenum carbide particles applied to an alumina surface.

FIG. 9 shows water gas shift reaction rates with different catalysts.

FIG. 10 compares water gas shift reaction rates vs. loading of active metal on the catalyst.

FIG. 11 shows water gas reaction rates vs. weight fraction of Pt.

FIG. 12 shows a model of Pt(111) layer (top) and Pt (epitaxial) layer (bottom) on molybdenum carbide.

FIG. 13 shows a graph of nominal loading vs. actual loading of Pt on catalyst.

FIG. 14 show a graph of hydrogen TPR for Pt—Mo₂C/Al₂O₃ catalysts.

FIG. 15 is a schematic of a catalyst synthesis scheme.

### DESCRIPTION

In one embodiment, a catalyst composition is made of a high surface area support particle (also called as a high surface area substrate or simply a substrate); so-called “islands” of an early transition metal interstitial compound attached to the high surface area support; and a metal disposed on part or all of the interstitial compound. When coverage by the metal is full or 100%, the catalysts can be called as having a core shell configuration or as being a core shell catalyst. In various embodiments, the high surface area support particle is selected from, alumina, silica, carbon, titania, and zeolites; the interstitial compound is a hydride, boride, carbide, or nitride of an early transition metal selected from Ti, Zr, Hf, V, Nb, Ta, Mo, and W; and the metal is selected from Cu, Ru, Rh, Ir, Ni, Pd, Pt, Ag, and Au.

The loading of the metal in the catalyst composition is such that from about 5% to 100% of the surface of the islands is covered with the metal. In some applications, catalysts ware preferred that contain10% coverage or greater, up to 100%. In preferred embodiments, the islands themselves are nanosized, meaning that they have dimensions in the sub-micrometer range. Typically, the islands (resulting from application of molybdates or other metal oxides onto the surface of the high surface area support or substrate) are on the order of 0.5-20 nm, and in exemplary embodiments from about 1 to about 10 nm in size (measured for example by scanning transmission electron microscopy.

In this way, a catalyst having a suitably high surface concentration of active metal (such as Pt) is provided, while keeping the cost to a minimum since the whole catalyst composition contains only a small of expensive metal. Examples of the loadings of the active metals in the catalysts can be calculated from the synthesis examples provided herein.

In preferred embodiments, the interstitial compound is a carbide such as VC, Mo₂C, or WC. In other embodiments, the active metal is Pt, Pd, or Cu. Non-limiting examples of preferred supports or substrates include carbon and alumina. In a particular embodiment, the (active) metal is Pt, the interstitial compound is Mo₂C, and the high surface area support is alumina. For shorthand, such a catalyst is designated as a Pt—Mo₂C/Al₂O₃ catalyst. Other catalysts can be analogously named by designating in order the active metal, the interstitial compound, and the nature of the substrate.

When such a composition is provided that has essentially 100% coverage of the interstitial compound by the active metal, there is provided a Pt—Mo₂C/Al₂O₃ core shell catalyst according to the invention.

In another embodiment, the invention provides a method of synthesizing such a catalyst composition. The method involves depositing an ionic species of a metal onto a core comprising an interstitial compound applied to a high surface area substrate under conditions where the ionic species is subsequently reduced in situ to the zero valent state on the surface of the interstitial compound. As with the catalyst composition, in the method the metal is selected from the group consisting of Cu, Ru, Rh, Pd, Ag, Os, Ir, Pt, and Au; the interstitial compound is selected from vanadium carbide, vanadium nitride, molybdenum carbide, molybdenum nitride, tungsten carbide, and tungsten nitride, and the high surface area substrate is selected from alumina, silica, carbon, titania, and zeolites.

In a preferred embodiment, the high surface area support is alumina or carbon. In these and other preferred embodiments, the interstitial compound is preferably a carbide such as VC, Mo₂C, or WC. In these and other other embodiments, the active metal is selected from Pt, Pd, and Cu.

Synthetic conditions are illustrated in the examples discussed below. In one embodiment, depositing the ionic species involves contacting the support with an aqueous solution of a metal precursor comprising the ionic species of the metal. The ionic species is a cationic metal species or an anionic metal species.

As with the composition, in a preferred method the metal is platinum, the interstitial compound is molybdenum carbide, and the high surface area support particle is alumina.

The invention also provides a catalyst composition made by one of the methods. For example, a Pt—Mo₂C/Al₂O₃ core shell catalyst is made by such a process.

The compositions and the products of the methods are useful as catalysts for the water gas shift reaction (WGS) and the Fischer-Tropsch synthesis reaction (FTS). Thus in one embodiment, the invention provides a method of producing hydrogen from reactants comprising carbon monoxide and water, the method comprising running a water gas shift reaction in the presence of such catalysts.

In another embodiment, the invention provides a method of synthesizing hydrocarbons from reactants comprising hydrogen and carbon monoxide, the method comprising running a Fischer-Tropsch synthesis reaction in the presence of such catalysts.

The present technology provides catalytic systems that include supports and active catalytic sites that can be optimized for reactions including the water gas shift reaction and the Fischer-Tropsch process. Such systems include different configurations of metal carbides and/or metal nitrides in conjunction with thin layers of noble metals adhered to the surface of the metal carbide and/or nitride compositions, the whole being applied to a high surface area support or substrate. These systems can be evaluated under experimental conditions and subjected to computational algorithms to guide the interpretation of experimental findings and correlate them to key features responsible for enhanced catalytic behavior.

Features and benefits of the present technology include: (1) catalyst formulations that are among the most active known for the water gas shift reaction; (2) methods for the synthesis of carbide and nitride supported catalysts with high surface metal utilizations (particularly useful for noble metal based catalysts); and (3) synthesis strategies for novel core-shell catalysts, an important new class of materials designed to maximize utilization of expensive metals.

In some embodiments, a catalyst composition comprises a catalyst support material having deposited thereon elemental metals, the metal being a catalytically active metal that can be reduced to the zero valent state by the interstitial compound provided on the catalyst support. In various embodiment, the metal is selected from noble metals and base metals such as (in order of increasing molecular weight) Cu, Ru, Rh, Pd, Ag, Os, Ir, Pt, and Au. Among these, Pt, Pd, and Cu are preferred in some embodiments. The catalyst support material onto which the active metal is deposited has an interstitial compound supported on a high surface area support or substrate. The interstitial compound is selected from early transition metal carbides, -nitrides, -borides, or hydrides. Examples of early transition metals suitable in various embodiments include V, Nb, Ta, Mo, and W. Among these, V, Mo, and W are preferred in some embodiments. Molybdenum carbide (Mo₂C) is a preferred interstitial compound in some embodiments. Combinations of interstitial compounds can also be used.

In some aspects, a metal precursor is used to form one or more thin layers of metal(s) on the surface of the support material, more specifically on the interstitial compound that sits on the surface of the substrate. Pd(NH₃)₄(NO₃)₂, H₂PtCl₆, and Cu(NO₃)₂ are non-limiting examples of precursors. The precursors contain an anionic (e.g. PtCl₆²⁻) or a cationic (e.g. Pd(NH₃)₄²⁺, Cu²⁺) metal species. For example, chloroplatinic acid is used as a precursor to an elemental platinum thin layer(s) adhered to the support upon treatment. The presently disclosed catalyst systems and compositions are particularly useful for carrying out reactions generally related to the water gas shift reaction (WGS) and to the Fischer-Tropsch Synthesis (FTS) process.

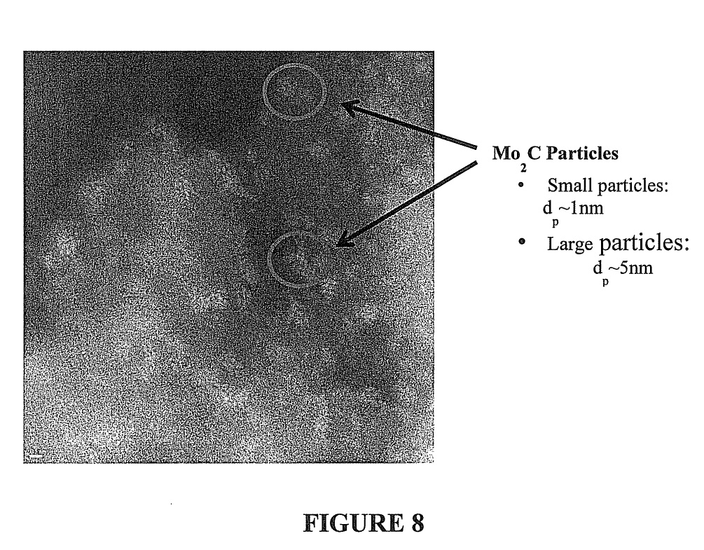

The catalysts can be constructed by first forming the interstitial compound on the surface of the substrate and then adding a metal layer to the surface of the interstitial compound. In general, a solution of a metal oxide (corresponding to the early transition metal of the interstitial compound) is contacted with particles of the substrate, such as by the incipient wetness technique illustrated in the Examples. The nature of the substrate surface and of the method and amount of addition are such that the substrate is not fully covered with the oxide. Instead, the oxide forms many nanosized islands on the surface of the substrate. The islands of oxides are next subjected to carburization or nitridation conditions, for example, to make interstitial carbides or nitrides on the surface of the substrate. The carbide or nitride interstitial compounds have the same footprint on the surface of the substrate as the oxides originally applied. The size of the islands is quite small, being on the order of nanometers (e.g. 0.5-20 nm, 1-10 nm, 1-5 nm, and so on. The result is that the interstitial compounds have a high surface exposed, since a high proportion of the applied early transition metal is found on the surface of the nanosized islands. The size of the islands and the dispersal across the surface of the substrate are illustrated in FIG. 8, where the white features are the interstitial “islands” and the white bar in the lower left of the micrograph shows 0.5 nm.

Metal oxide is added, by incipient wetness or other technique, to the surface of the substrate at a level to provide suitable coverage of the active metal that is later added to the interstitial compound prepared from the oxide/substrate precursor. Suitable coverage for a given application will be determined by the nature of the substrate, interstitial compound, active metal, and by the type of reaction and amount of catalyst required for the application, and other factors. Suitable levels of oxide coverage on the substrate can be determined empirically and applied in individual situations. A convenient and easy method to calculate unit for coverage is the number of moles of oxide applied per unit surface of the substrate. As a starting rule of thumb, suitable coverage can be selected, for example, from 0.01-100 μmol per square meter of BET surface on the substrate. In some embodiments, oxide is added to the substrate to provide coverage of 0.1-50, 0.5-30, 1-20, and 1-10 μmol per square meter. In some embodiments, going to a lower value could result in a catalyst having insufficient active metal to be useful in the intended catalytic application. On the other hand, sometimes higher levels could be undesirable because of cost or other concerns. Alternatively, target loadings can be calculated in terms of weight % oxide based on the weight of the substrate used, once the BET surface area of the substrate is known. Further non-limiting description is given in the Examples section.

An active metal is applied to the catalyst support made of the interstitial compounds on the surface of the substrate. In general, the catalyst support is exposed to an aqueous solution of a metal precursor as described above. By a wet impregnation method, the precursor metal species is applied to the catalyst support. The precursor species attaches to and reacts preferentially with the interstitial compound, not the exposed substrate surface. At the same time, the metal of the interstitial compound is believed to act as reducing agent to transform the active metal of the precursor to the zero valent state to form catalyst compositions of the invention.

Wet impregnation proceeds according to basic principles of wetting and ionic attraction. A point of zero charge (PZC) can be measured of the Mo₂C (or other interstitial support. Adjust water to an initial pH. Add unpassivated Mo₂C to the water, observe pH over time until equilibrates at a new pH. Plot final pH as a function of initial pH and observe a flat portion of the curve where final pH=PZC. To add metal from a metal precursor to the Mo₂C, a solution of a metal precursor is used characterized by a pH. When the solution pH is below the PZC of the support material, the surface of the support becomes protonated, has a net positive charge, and attracts anionic metal complexes (e.g. [PtCl₆]₂—). For solution pH above the PZC, the surface becomes deprotonated, has a net negative charge, and attracts cationic metal complexes ([(NH₃)₄Pt]₂₊). It is believe that controlling the pH and selecting the appropriate metal precursor along these guidelines is helpful to producing suitable catalyst compositions.

If desired, ICP-OES or other analytical technique can be used to follow the solution concentration of metal over time, to determine the point at which the maximum amount of metal has been deposited on the Mo₂C precursor.

In various embodiments, the active metal particles formed via the wet impregnation of interstitial compound (e.g. molybdenum carbide) with metal precursor are anisotropic. The x- and y-dimensions of the particles (on the two dimensional surface) are much longer than the z-dimension. Based on their shape, these particles appear to be “raft-like”, thus resulting in a high surface area to volume ratio and a high density of active interface sites.

It will be understood that supported metal catalysts can be prepared by depositing noble or other metals by various chemical and physical methods including but not limited to impregnation, plating, metallo-organic decomposition, chemical vapor deposition, plasma assisted vapor deposition, physical vapor deposition, sputtering, laser ablation, plasma discharge emission, and molecular beam epitaxy among others. It is to be understood that these examples are provided for illustrative purposes and are not to be construed as limiting the scope of the present disclosure.

Another aspect of the present technology is to obtain a high dispersion of noble metal over the support where the perimeter of the noble metal area is much larger than its volume, or put another way, where the two dimensional extent of the noble metal is much greater than its dimension in the third direction, being a thickness of the applied noble metal. The noble metal is thus applied in thin layers and a large proportion of the noble is active at the surface to participate in catalytic reactions. This exposes a high percentage of surface area for catalyst purposes, which is generally advantageous. This is achieved by taking advantage of the metallic properties of transition metal interstitial compounds and depositing “raft-like” structures of noble metal onto these supports. This is in contrast to metals deposited on most oxide surfaces, which tend to lead to the formation of spheres, beads, or other geometrical structures where the surface area to volume ratio is low.

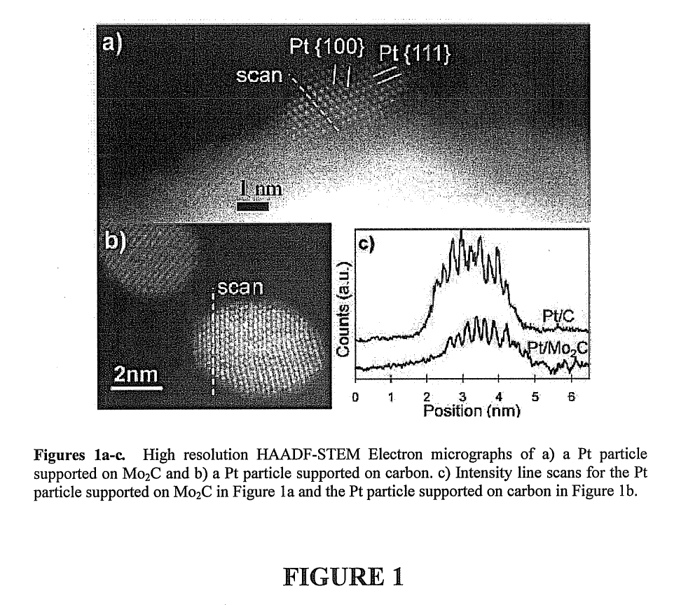

Arrhenius plots of the WGS reaction rates are shown in FIG. 1a for the 2.7% Pt/Al₂O₃, 5% Pt/CeO₂, 2% Pt/TiO₂, and 4% Pt/Mo₂C catalysts. The conversions were limited to 10% to avoid transport limitations. The reactant was designed to simulate the composition of effluent from a partial oxidation reformer and contained 11% CO, 21% H₂O, 43% H₂, 6% CO₂ and 19% N₂. The Pt/Mo₂C catalyst exhibited the highest rates while the Pt/Al₂O₃ catalyst was the least active. Rates and apparent activation energies for the oxide supported catalysts were within the range of those reported in the literature (Table 1), although the reaction conditions were slightly different from those used in our work. The Mo₂C support alone exhibited a gravimetric rate of 49 μmol/gcat·s at 240° C. compared to 227 μmol/gcat·s for the 4% Pt/Mo₂C catalyst. Under the same conditions, a rate of 54 μmol/gcat·s was measured for a commercial Cu—Zn—Al WGS catalyst. These results suggest that Mo₂C supported metals are promising WGS catalysts.

Referring now to the several figures, additional aspects of the present technology are illustrated.

FIG. 1a shows high angle annular dark field scanning transmission electron microscopy (HAADF-STEM) micrographs. A high resolution image of one of the particles (FIG. 1a) illustrates the Pt crystallographic planes, and reveals a relatively low contact angle between the Pt particle and Mo₂C support. A low contact angle is indicative of strong interactions between Pt and the Mo₂C surface. FIGS. 1b-c show that intensity line scans (FIG. 1c) for a number of particles on the Pt/Mo₂C catalyst were relatively flat compared to the rounded cubo-octahedral particles on a Pt/C catalyst with similar particle sizes (FIG. 1b).

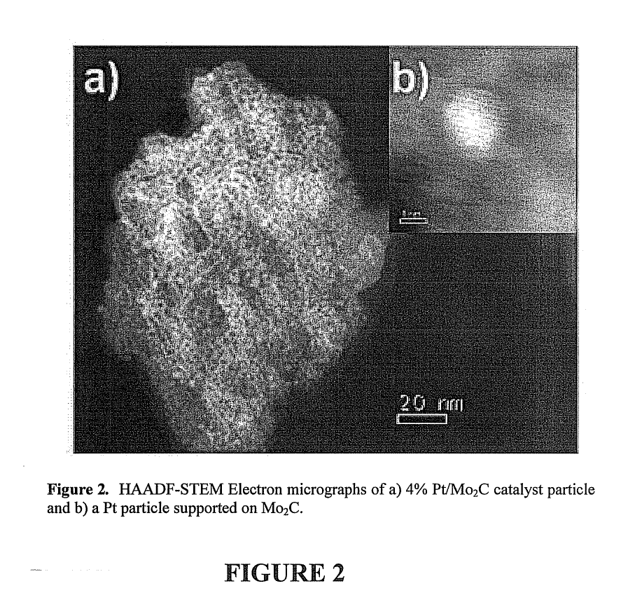

FIG. 2 shows high angle annular dark field scanning transmission electron microscopy (HAADF-STEM) with Cs-correction to directly examine the particle sizes and shapes. The bright spots in FIG. 2a (amplified in FIG. 2b) correspond to Pt particles, as confirmed by x-ray energy dispersive spectroscopy. Most of these Pt particles were approximately 2-4 nm in size. This relative uniform distribution of nanoscale particles is partly due to strong interactions between the support and Pt precursor.

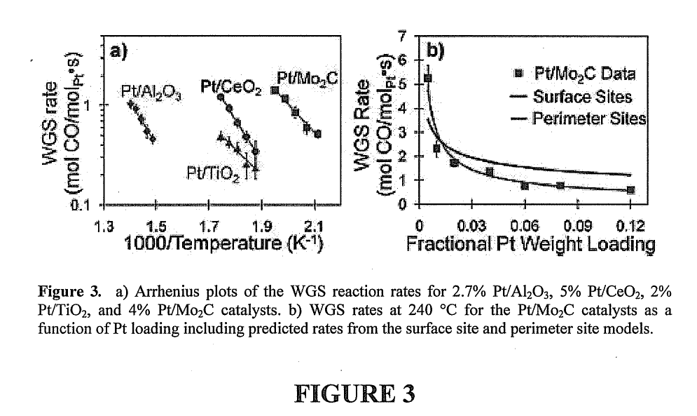

FIG. 3a shows Arrhenius plots of the WGS reaction rates for 2.7% Pt/Al₂O₃, 5% Pt/CeO₂, 2% Pt/TiO₂, and 4% Pt/Mo₂C catalysts. FIG. 3b shows WGS rates at 240° C. for the Pt/Mo₂C catalysts as a function of Pt loading, including predicted rates from the surface site and perimeter site models.

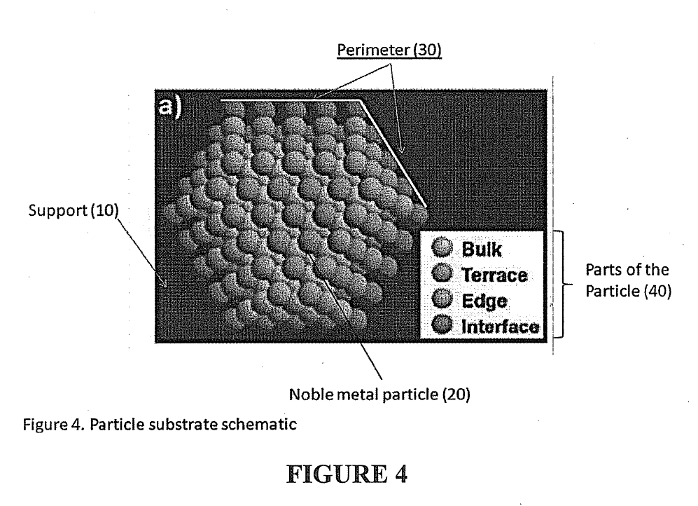

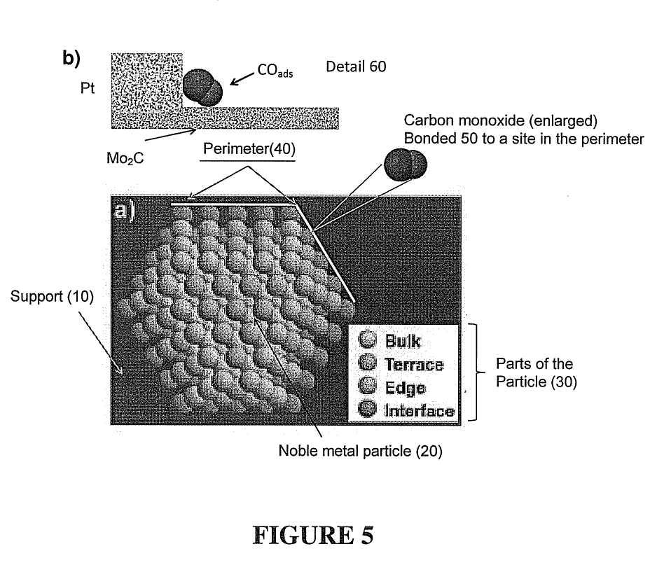

FIG. 4 shows a particle (20) sitting on the surface of a substrate or support (10) showing various facets (30) of the particle (20) on the support (10) including the perimeter (40) of the particle (20) on the support (10). There is evidence that the most reactive sites for reactions including WGS are at the particle perimeter (interface between particle and support). FIGS. 5a-b show a particle (20) sitting on the surface of a substrate or support (10) showing various parts (30) of the particle (20) on the support (10) including the perimeter (40) of the particle (20) on the support (10) including a molecule of carbon monoxide bonded (50) onto a site (detail 60) located on a step in the perimeter 40 of the particle 20. The interface sites, illustrated in FIG. 5, allow the CO to interact with both Pt atoms and the Mo₂C surface. This type of interface site appears to be the active site for the WGS reaction and results in a catalyst with high WGS activity per unit Pt.

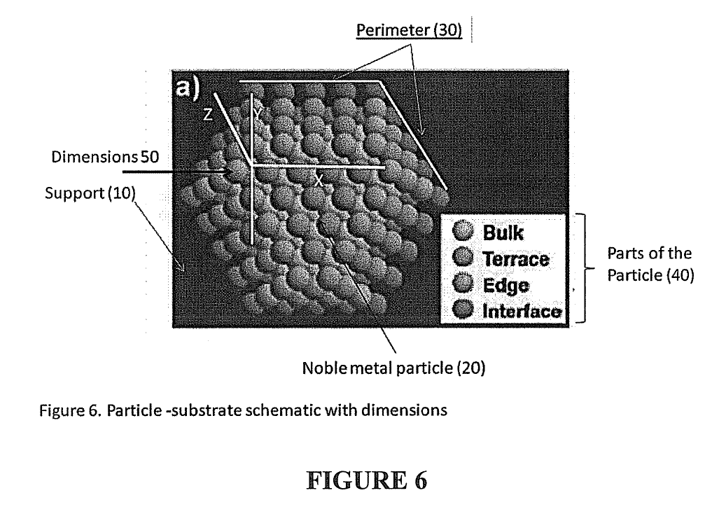

FIG. 6 shows a particle (20) sitting on the surface of a substrate or support (10) showing various parts (30) of the particle (20) on the support (10) including the perimeter (40) of the particle (20) on the support (10) in three dimensions (50) represented by the -x, -y, -z planes. The Pt particles formed via the wet impregnation of Mo₂C with chloroplatinic acid were anisotropic. The x- and y-dimensions of the particles were much longer than the z-dimension. Based on their shape, these particles appear to be “raft-like”, thus resulting in a high surface area to volume ratio and a high density of active interface sites.

To further illustrate the present disclosure, the following examples are given. It is to be understood that these examples are provided for illustrative purposes and are not to be construed as limiting the scope of the present disclosure.

### Example 1

Pt loadings from about 0.5 to 12 wt % were deposited onto Mo₂C supports. Loadings for the oxide supported catalysts were controlled to achieve the same Pt surface coverage as that for the 4% Pt/Mo₂C catalyst (1.1 atom/nm²). It will be understood by one skilled in the art that the Pt loading range may extend below 0.5 wt % and beyond 12 wt %. The results of the corresponding WGS reactions are presented in Table 1.

TABLE 1
WGS rates and activation energies for supported Pt catalysts.

| | Pt | | | WGS Rate^{c} | |
|---|---|---|---|---|---|
| | Loading | E_{App}^{a} | | (mol | |
| Catalyst | (wt %) | (kJ/mol) | Tᵣₓₙ^{b} (° C.) | CO/mol_{Pt} · s) | Ref. |
| Pt/Mo₂C | 3.9 | 53 | 240 | 1.423 | This paper |
| Pt/CeO₂ | 5.0 | 80 | 260 | 0.346 | This paper |
| Pt/CeOₓ | 1.2 | 83 | 250 | 0.093^{d} | (20) |
| Pt/CeO₂ | 0.49 | — | 250 | 0.081^{e} | (12) |
| Pt/TiO₂ | 2.0 | 51 | 260 | 0.236 | This paper |
| Pt/TiO₂ | 0.5 | 66 | 250 | 0.402^{f} | (11) |
| Pt/TiO₂ | 0.52 | — | 250 | 0.154^{e} | (12) |

^{a}Apparent activation energy
^{b}Temperature for reaction rate measurement
^{c}Rates for all catalysts were collected at atmospheric pressure
^{d}Feed composition: 11% CO, 8% CO₂, 26% H₂, 26% H₂O, bal He
^{e}Feed composition: 4.4% CO, 8.7% CO₂, 28% H₂, 29.6% H₂O, 0.1% CH₄, bal N₂
^{f}Feed composition: 3% CO, 6% CO₂, 20% H₂, 10% H₂O, bal He

In Table 1, reference (20) is Pierre, D.; Deng, W.; Flytzani-Stephanopoulos, M. Top. Catal. 307 2007, 46, 363-373. Reference (12) is Gonzalez, I. D.; Navarro, R. M.; Wen, W.; Marinkovic, 290 N.; Rodriguez, J. A.; Rosa, F.; Fierro, J. L. G. Catal. Today 2010, 149, 291 372-379. And reference (11) is Panagiotopoulou, P.; Kondarides, D. Catal. Today 2007, 127, 319-329.

In yet another aspect within the scope of example 1, the noble metal loading and/or thermal treatment schedules can be varied in order to achieve core-shell catalyst configurations. Furthermore, theoretical calculations can be used to suggest various metal/solute concentrations for the interstitial material and for noble metal loading on the surface of the interstitial compound.

### Example 2

In this example, a preparation method comprises the use of an oxygen-free aqueous solution of noble metal salt according to the following steps. A temperature-programmed reaction method was used to synthesize high surface area Mo₂C and Mo₂N supports. This method involves reacting bulk or supported Mo oxides with a mixture of 15% CH₄ in H₂ or NH₃ in a quartz reactor as the temperature is increased linearly. The appropriate reaction temperatures were determined based on results from thermogravimetric analysis. The resulting carbides or nitrides were carefully transferred from the synthesis reactor, without passivation or air-exposure, to vessels containing the deaerated metal salt solution. The metal concentration was adjusted to achieve the desired loading. Argon was bubbled through the solutions continuously to prevent the dissolution of O₂. The mixtures were typically held at room temperature for 2 hrs and at 40° C. for 1 hr with occasional stirring. The product was then cooled to ambient temperature. The liquid solution was retained for analysis of residual metal content. The remaining slurry was placed inside a quartz reactor under Ar then dried in flowing H₂ at 110° C. for 2 hrs and reduced at 450° C. for 4 hrs. After reduction, the material was cooled to ambient temperature and passivated with a mixture of 1% O₂ in He for 5 hrs.

### Example 3

### Pt—Mo₂C/Al₂O₃ Catalyst preparation

A Mo₂C/Al₂O₃ catalyst support was synthesized using a temperature programmed reaction procedure. Molybdate was deposited onto the Al₂O₃ high surface area support via incipient wetness of Al₂O₃ with an aqueous solution containing ammonium paramolybdate (AM, (NH₄)₆Mo₇O₂₄.4H₂O, 81-83% MoO₃, Alpha Aesar) followed by drying at 110° C. for 12 h. Approximately 1 g of the resulting AM/Al₂O₃ catalyst support precursor was loaded into a quartz tube reactor on top of a quartz wool plug. The AM/Al₂O₃ was reduced and carburized in 15% CH₄/H₂ flowing at 210 mL/min as the temperature was increased from room temperature (RT) to 200′C (heating rate of 10° C./min), and then the temperature was increased from 200° C. to 590′C at a rate of 1′C/min. The final temperature was maintained for 2 h before quenching the material to RT. The resulting material was passivated using a 1% O₂/He mixture with a flow rate of 20 mL/min for at least 5 h.

A Mo₂C—Al₂O₃ supported Pt catalyst was prepared via a wet impregnation method. The freshly synthesized Mo₂C/Al₂O₃ was transferred under Ar to a deaerated, aqueous solution containing an appropriate amount of chloroplatinic acid (H₂PtCl₆.6H₂O, 99.95% metal basis, Alfa Aesar) to achieve the desired loading. After 3 h loading, the slurry was dried at RT in H₂ and reduced in H₂ at 450° C. Finally, the sample was passivated at RT using a 1% O₂/He gas mixture.

### Example 4

### Catalyst Evaluation

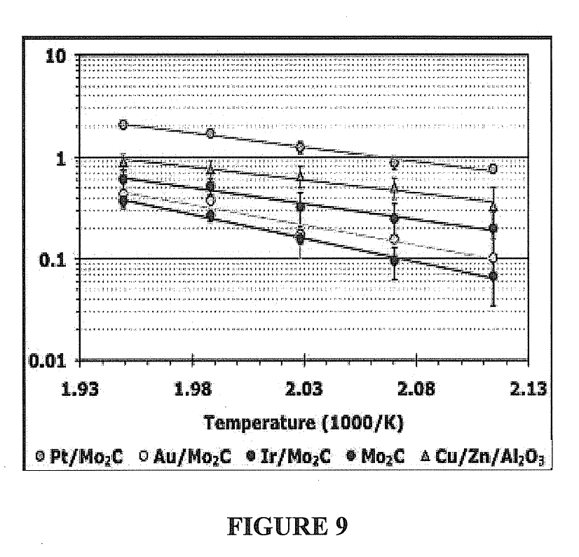

Prior to the WGS and FTS reaction rate and selectivity measurements, the catalysts were pretreated in a mixture of 15% CH₄ in H₂, NH₃ or H₂ for 4 hrs at temperatures defined based on results from temperature programmed reduction analysis. The WGS rates were measured at atmospheric pressure and temperatures of 200-240° C. using a feed consisting of 9% CO, 6% CO₂, 30% H₂O, 39% H₂, and the balance N₂. The gas hourly space velocity (GHSV) ranged from 75,000 to 150,000 h⁻¹ based on the total flow rate. The exiting gas mixture was passed through a condenser maintained at 0° C. to remove H₂O, and the composition was analyzed online using a gas chromatograph equipped with a thermal conductivity detector (TCD). FIG. 9 compares WGS rates (the unlabeled ordinate) for the, Au/Mo₂C, Ir/Mo₂C, Cu/Zn/Al₂O₃ (currently used in industrial processes), and, catalysts (from bottom to top in the Figure, so that Mo₂C is the least and Pt/Mo₂C is the most active). Addition of the metals resulted in a significant increase in the WGS rate. Increased reaction rates typically translate into reduced reactor sizes and operating costs. The Pt/Mo₂C catalyst was the most active with rates that exceeded those of the Cu/Zn/Al₂O₃ catalyst.

### Example 5

### Catalyzed Water Gas Shift Reaction as Function of Pt Surface Coverage

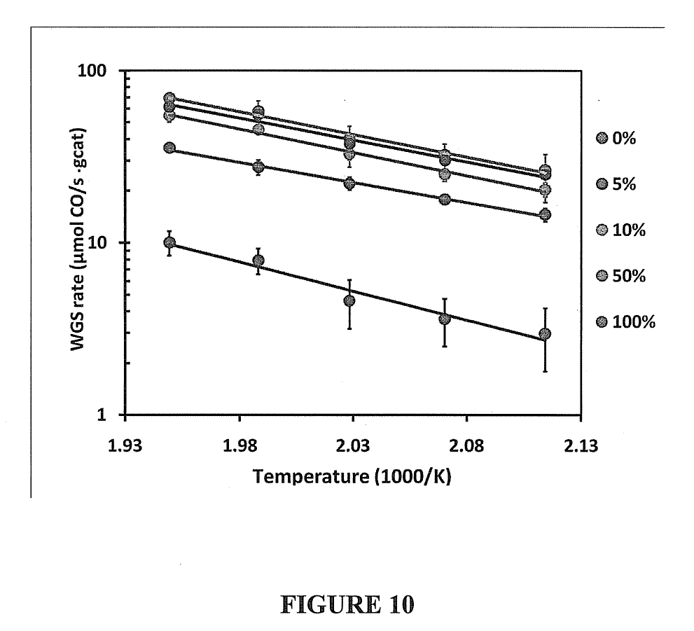

FIG. 10 shows WGS rates for the Pt—Mo₂C/Al₂O₃ catalysts as a function of Pt surface coverage. The curves from bottom to top are those for 0%, 5%, 10%, 50%, and 100% loading. The gravimetric reaction rates increased with increasing Pt surface coverage. The overall reaction rates for the Pt—Mo₂C/Al₂O₃ catalysts (35-70 μmol CO/g·s at 240° C.) were lower than the rates for the Pt/Mo₂C catalysts due to the lower surface site densities. The activation energies were in the range of 44-59 kJ/mol and were similar to those for the Pt/Mo₂C catalysts.

### Example 6

### Water Gas Shift Reactions

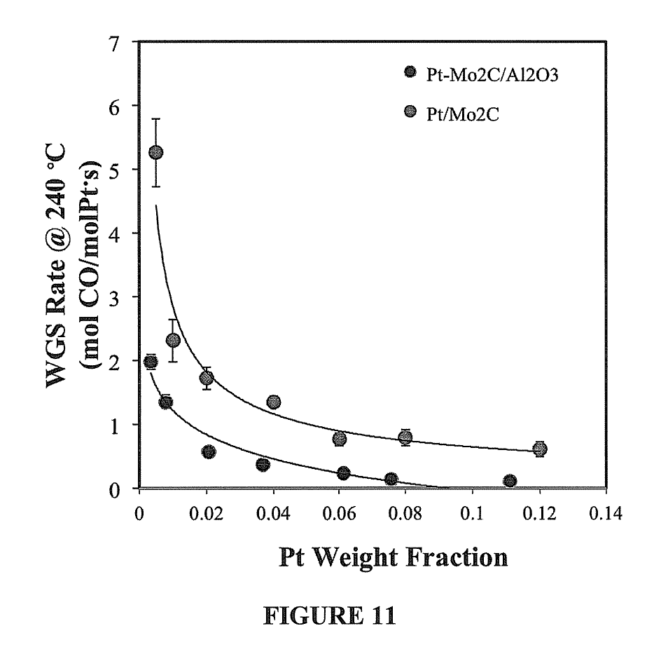

FIG. 11 compares WGS rates for the Pt/Mo₂C (upper curve) and Pt—Mo₂C/Al₂O₃ (lower curve) catalysts with varying Pt weight loadings. The availability of active sites appears to decrease with increased loading. The trend for the Pt—Mo₂C is consistent with the active sites residing at the interface between the Pt particles and Mo₂C support. This result also suggests that relatively low Pt loadings could be used for these catalysts. Rates for the Pt—Mo₂C/Al₂O₃ catalysts exhibited a trend that was similar to that for the Pt/Mo₂C catalysts, suggesting that Pt interacted primarily with the Mo₂C surface instead of Al₂O₃. When adjusted for Mo₂C coverage the trendlines converge.

### Example 7

### Characteristics of Pt/Mo₂C Catalyst

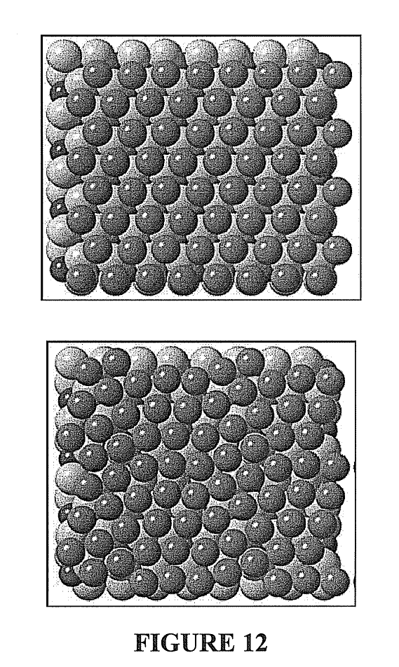

Characteristics of the Pt/Mo₂C catalysts were investigated via density functional theory (DFT) calculations. FIG. 12 shows the structure of a relaxed Pt(111) layer (top) and an epitaxial Pt layer (bottom, Pt occupying Mo hollow sites) on the surface of Mo₂C. Relative to the cohesive energy, binding energies were −0.8 eV/Pt atom for the Pt(111) structure and −1.0 eV/Pt atom for the epitaxial structure. These strong interactions are consistent with the HRTEM and XAS results that Pt forms “raft-like” structures on the surface of Mo₂C.

### Example 8

### Pat Loading on Catalysts

The strong Pt—Mo₂C interactions reported in Example 7 suggested that Mo₂C would be an effective “core” for Pt “shells” in a core-shell configuration. Catalysts were prepared using a wet impregnation method at room temperature for 3 h using a range of nominal loadings. A plot of measured loading vs. nominal loading for the Pt—Mo₂C/Al₂O₃ catalysts shows a good parity with the nominal loading at a low metal loading. However, when the metal loading is higher (the change is observed at about 6-8% loading), the measured loading is lower than the nominal loading. The turning point between 6% and 8% may indicate that the surface of Mo₂C is saturated with Pt. To achieve higher loadings, higher temperatures were employed during preparation of the catalyst. ICP-OES measurements show that using higher temperatures enhanced Pt deposition.

### Example 9

### Diffraction Patterns for Catalysts

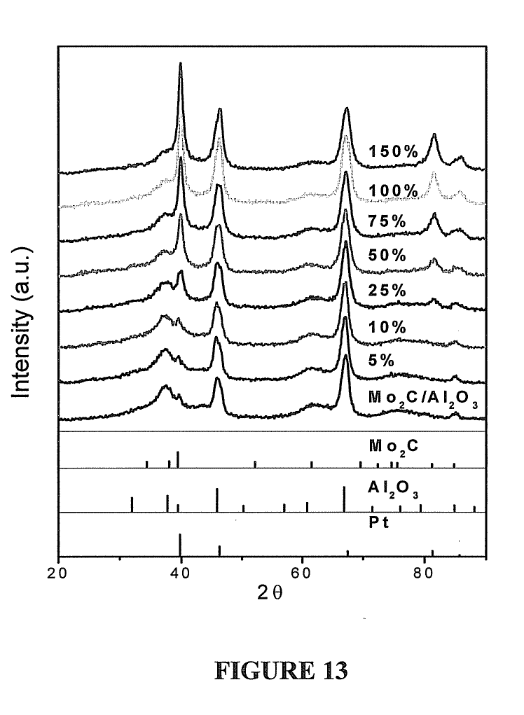

Diffraction patterns for the Pt—Mo₂C/Al₂O₃ catalysts are presented in FIG. 13. The absence of the peaks for Mo₂C in the Mo₂C/Al₂O₃ pattern indicates that the Mo₂C was highly dispersed on the Al₂O₃. When the Pt loading is higher than 2 wt %, evidence for crystalline Pt was observed.

### Example 10

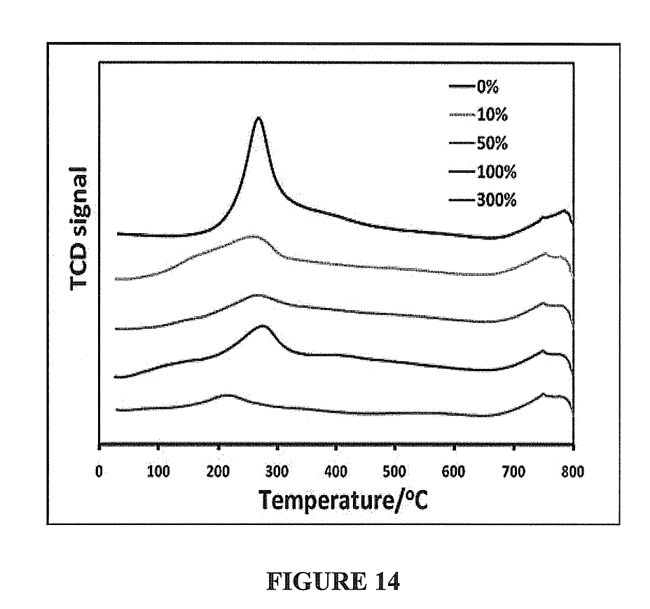

Hydrogen-TPR profiles for Pt—Mo₂C/Al₂O₃ catalysts with varying nominal surface coverages are shown in FIG. 14. The peak in the range of 100 to 150° C. was attributed to the reduction of Pt, while the peak in the range of 200 to 300° C. results from the removal of oxygen from the Mo₂C surface (formed during the passivation process). The intensity of the reduction peak for the oxygen in the passivation layer decreased with increasing Pt surface

### Example 11

### Core Shell Catalyst Preparation

Mo₂C/Al₂O₃, Mo₂C/C and VN/C catalyst supports were synthesized using a temperature programmed reaction procedure to apply an interstitial compound to a substrate. The molybdate was deposited onto the Al₂O₃ support (or carbon black support) via incipient wetness of Al₂O₃ (or carbon black support) with an aqueous solution containing ammonium paramolybdate (AM, (NH₄)₆Mo₇O₂₄.4H₂O, 81-83% MoO₃, Alpha Aesar). The γ-Al₂O₃ (BET surface area 120 m²/g, pore volume 0.5 ml/g, Alfa Aesar) and carbon black (BET surface area 200 m²/g, pore volume 0.3 ml/g, Cabot Corporation) with a particle size range of 60-120 mesh were employed. Typically, 5 ml AM solution with a concentration of 0.7 M was added dropwise into 10 g Al₂O₃ with drastic shaking. The resulting samples were dried at 110° C. overnight. V₂O₅ supported on carbon black was derived from vanadium gel. Typically, the V₂O₅ powder was dissolved in H₂O₂ (Fisher Chemicals) at O′C due to the high exothermicity of the reaction. The solution was stirred for about 1.5 h until no oxygen evolution from the solution. The carbon black was added under stirring. The as-made suspension was aged and the formed gel (V₂O₅.nH₂O/C) was then dried and grinded.

Approximately 1 g of AM/Al₂O₃ support (or AM/C) with a particle size range of 60-120 mesh was loaded into a quartz tube reactor on top of a quartz wool plug. The AM/Al₂O₃ (or AM/C) was reduced and carburized in 15% CH₄/H₂ flowing at 210 mL/min as the temperature was increased from room temperature (RT) to 200° C. (heating rate of 10° C./min), and then the temperature was increased from 200° C. to 590° C. at a rate of 1° C./min. The final temperature was maintained for 2 h before quenching the material to RT. The resulting material was passivated using a 1% O₂/He mixture with a flow rate of 20 mL/min for at least 5 h. Similarly, approximately 1 g V₂O₅/C of support with a particle size range of 60-120 mesh was loaded into the reactor. The V₂O₅/C was thermally treated under NH₃ at 600 mL/min as the temperature was increased from RT to 800° C. The final temperature was maintained for 2 h following with cooling down to RT.

The Mo₂C—Al₂O₃, Mo₂C—C and VN-C supported metal catalyst was prepared via a wet impregnation method. The freshly synthesized Mo₂C/Al₂O₃, Mo₂C—C and VN-C were transferred under Ar to a deaerated, aqueous solution containing the metal precursors. H₂PtCl₆, Cu(NO₃)₂, Ag(NO₃)₂, HAuCl₄ and RuCl₃ were employed as the metal precursors. For example, for the preparation of the Pt—Mo₂C/Al₂O₃ catalyst with a 3.9 wt % Pt loading which was corresponding to a 50% Pt coverage on Mo₂C surface, the chloroplatinic acid solution with a concentration of 1.3 mg/mL was prepared and deaerated prior to the freshly synthesized Mo₂C/Al₂O₃ transfer. The support powder was left in Pt solution for 3 h and periodically stirred with bubbling maintained. After decanting the excess solution, the material was loaded into a quartz reactor and dried in H₂ at 350 mL/min for 3 h at RT. Subsequently, the temperature was increased to 110′C in 1 h and held there for 2 h. The temperature was then increased to 450′C in one hour and held for 4 h. Finally, the material was quenched to RT and passivated in a 1% O₂/He mixture at 20 mL/min for at least 5 h.

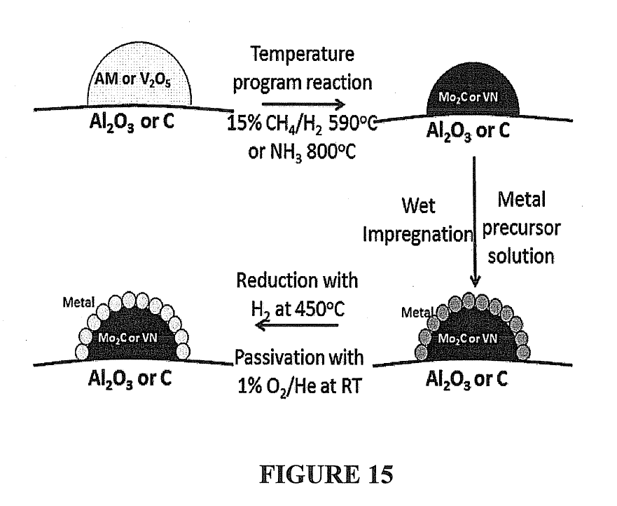

FIG. 15 is a schematic illustration of the synthesis here described.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

What is claimed is:

1. A catalyst composition comprising:
a high surface area support particle;
islands of an early transition metal interstitial compound attached to the high surface area support; and
an active metal disposed on part or all of the interstitial compound,
wherein
the high surface area support particle is selected from alumina, silica, carbon, titania, and zeolites,
the interstitial compound is a hydride, boride, carbide, or nitride of an early transition metal selected from the group consisting of Ti, Zr, Hf, V, Nb, Ta, Mo, and W, and
the active metal is selected from Cu, Ru, Rh, Ir, Ni, Pd, Pt, Ag, and Au.

2. A catalyst according to claim 1, wherein loading of the early metal transition interstitial compound is 0.01-100 μmol per square meter BET surface of the high surface area support.

2. A catalyst according to claim 1, wherein loading of the early metal transition interstitial compound is 0.1-10 μmol per square meter BET surface of the high surface area support.

4. A catalyst composition according to claim 2, wherein loading of the active metal gives 10% to 100% coverage of the surface of the interstitial compound.

5. A catalyst composition according to claim 2, wherein loading of the active metal gives 10% to 90% coverage of the surface of the interstitial compound.

6. A catalyst composition according to claim 2, wherein loading of the active metal gives 100% coverage of the surface of the interstitial compound.

7. A catalyst according to claim 1, wherein the interstitial compound is VC, Mo₂C, or WC, and the metal is Pt, Pd, or Cu.

8. A catalyst according to claim 7, wherein the high surface area support is carbon or alumina.

9. A catalyst according to claim 8, wherein the noble metal is Pt, the interstitial compound is Mo₂C, and the high surface area support is alumina.

10. A Pt—Mo₂C/Al₂O₃ core shell catalyst.

11. A core shell catalyst according to claim 10, wherein Al₂O₃ is a substrate characterized by a BET surface area, and the catalyst comprises 0.01-100 μmol Mo₂C per square meter of the surface area.

12. A core shell catalyst according to claim 10, wherein Al₂O₃ is a substrate characterized by a BET surface area, and the catalyst comprises 0.1-10 μmol Mo₂C per square meter of the surface area.

13. A method of synthesizing a catalyst composition, the method comprising depositing an ionic species of a metal onto a core comprising an interstitial compound applied to a high surface area substrate under conditions where the ionic species is subsequently reduced in situ to the zero valent state on the surface of the interstitial compound,
wherein
the metal is selected from the group consisting of Cu, Ru, Rh, Pd, Ag, Os, Ir, Pt, and Au,
the interstitial compound is selected from vanadium carbide, vanadium nitride, molybdenum carbide, molybdenum nitride, tungsten carbide, and tungsten nitride, and
the high surface area substrate is selected from alumina, silica, carbon, titania, and zeolites.

14. A method according to claim 13, wherein the high surface area support is alumina or carbon.

15. A method according to claim 13, wherein depositing comprises contacting the support with an aqueous solution of a metal precursor comprising the ionic species of the metal.

16. A method according to claim 15, wherein the ionic species is a cationic noble metal species.

17. A method according to claim 15, wherein the ionic species is an anionic noble metal species.

18. A method according to claim 13, wherein the noble metal is platinum, the interstitial compound is molybdenum carbide, and the high surface area support particle is alumina.

19. A Pt—Mo₂C/Al₂O₃ core shell catalyst made by a process according to claim 18.

20. A method of producing hydrogen from reactants comprising carbon monoxide and water, the method comprising running a water gas shift reaction in the presence of a catalyst made by a process according to claim 13.

21. A method of producing hydrogen from reactants comprising carbon monoxide and water, the method comprising running a water gas shift reaction in the presence of a catalyst made by a process according to claim 19.

22. A method of synthesizing hydrocarbons from reactants comprising hydrogen and carbon monoxide, the method comprising running a Fischer-Tropsch synthesis reaction in the presence of a catalyst made by a process according to claim 13.

23. A method of synthesizing hydrocarbons from reactants comprising hydrogen and carbon monoxide, the method comprising running a Fischer-Tropsch synthesis reaction in the presence of a catalyst made by a process according to claim 19.
